(19) United States
(12) Patent Application Publication
(10) Pub. No.: US 2026/0201306 A1
(43) Pub. Date: Jul. 16, 2026

(54) SYSTEMS AND METHODS FOR OXYGENATING CULTURE MEDIA IN PERFUSION BIOREACTORS

(71) Applicant: CORNING INCORPORATED, CORNING, NY (US)

(72) Inventor: Vasiliy Nikolaevich Goral, Painted Post, NY (US)

(21) Appl. No.: 19/134,689

(22) PCT Filed: Nov. 22, 2023
(86) PCT No.: PCT/US2023/080868

§ 371 Date: May 30, 2025

Related U.S. Application Data
(60) Provisional application No. 63/428,975, filed on Nov. 30, 2022.

(51) Int. Cl. C12M 1/00; C12M 1/04; C12M 1/34

(52) U.S. Cl. CPC C12M 29/10; C12M 23/24; C12M 27/18; C12M 29/08; C12M 29/22; C12M 29/26; C12M 41/44

(57) ABSTRACT

A cell culture system is provided that includes a cell culture vessel enclosing an interior configured to culture cells in liquid cell culture media, and a media conditioning system. The media conditioning system includes a media conditioning vessel for conditioning liquid media, and an oxygenation column. The oxygenation column includes an enclosure housing an interior space, an oxygen-depleted media inlet fluidly connected to the interior space, an excess gas vent fluidly connected to the interior space, and a lower opening fluidly connected to the interior space. The lower opening is disposed at a lower end of the oxygenation column and in a fluid path between the interior space and the media conditioning vessel. The oxygenation column is designed to mix the oxygen-depleted media with a gas comprising oxygen in a counterflow manner, and thus achieve better dissolved oxygen saturation in the media.

### CROSS-REFERENCE TO RELATED APPLICATIONS

This application claims the benefit of priority under 35 U.S.C. § 119 of U.S. Provisional Application Ser. No. 63/428,975 filed on Nov. 30, 2022, the content of which is relied upon and incorporated herein by reference in its entirety.

### FIELD OF THE DISCLOSURE

This disclosure generally relates to media conditioning in cell culture bioreactor systems, and more particularly to media conditioning systems and methods for oxygenating cell culture media in perfusion bioreactor.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success. The process efficiency of a bioreactor cell culture should be high for economically viable production. In the area of bioreactor design, the bioprocessing industry is moving towards the development of high density cultures. For given production capacity, high density system are more compact in size and more cost effective.

However, there are limitations for increasing cell density and thus the reactor performance. These include operation, aeration and media formulation. In cell culture reactors, the cells must be grown under controlled conditions, including being suspended or perfused in a cell culture media, which is a liquid media containing nutrients necessary for cell life and growth. The contents of the cell culture media, including the pH and content of dissolved gases (including, e.g., dissolved oxygen), as well as the temperature of the media and/or the cells, must be controlled to optimize cell growth and performance of the bioreactor. Therefore, media conditioning systems are used in combination with or integrated into the bioreactors to condition the media therein. These media conditioning systems can, for example, control temperature, pH, carbon dioxide content, dissolved oxygen content, and other aspects of the media. Depending on the cells being grown or the stage of the culture process, the specific media conditioning needs can vary. Typically, media conditioning may be performed in a hollow container or vessel (e.g., a beaker or bottle) with a complicated system of probes to control the composition of the media, one or more stirrers to mix the media, and some type of temperature control jacket around the vessel. The probes may enter the vessel through a cap on the vessel body, and sterility is always a concern, especially if the system is open or opened during use.

Cells density in the reactor is often determined by oxygen delivery to the cells. Mass transfer rate from gas to the medium should balance the oxygen consumption by the cells. Currently there are several aeration methods used in bioprocessing industry. These include surface aeration, membrane aeration, macro sparging and micro sparging. Sparged cultures achieve higher oxygen transfer rates in comparison to other techniques. However, sparging rates are limited by cell lysis and foaming issues. One of the approaches to further increase oxygenation rate is described in U.S. Pat. No. 9,388,375, which suggests combining gas overlay and gas sparging process in the same reactor. U.S. Pat. No. 9,512,392 also discloses a method to increase dissolved oxygen (DO) in culture medium, which forms a foundation to design and make effective mammalian cell culture bioreactors. Historically, the bioprocessing industry was based on design principles of traditional microbial fermenters, which relied heavily on stainless steel technologies. Because of this, most sparge systems found in stirred tank bioreactors were not suitable for mammalian cell cultures. They relied on high-shear mixers to break up gas bubbles. However, mammalian cell culture requires a gentle mixing and lower gas shear rates, which require differently engineered sparges.

The ability to scale up a biomanufacturing process is essential for process development and the production of biotherapeutic agents. Bioreactor process set points, acceptable ranges, and general operating parameters used at the large scale are commonly based on those developed at the bench top or small scale where the cost is smaller. Scaling up bioreactor processes—especially oxygenation efficiency—is challenging. It is difficult, for example, to simultaneously maintain equivalent bioreactor characteristics such as bubble size, distribution, residence time, and comparable bubble surface area. Therefore, the mass transfer of gasses remains one of the critical and difficult parameters used for bioreactor control. Sufficient O₂ or air delivery is required not only to support cell growth, metabolism, and biopharmaceutical production, but also to control CO₂ accumulation in the media, which can negatively impact performance endpoints

Accordingly, there is a need for improved media conditioning systems that can meet the demands high density cell cultures in terms of gas-liquid mass transfer into the cell culture media.

### SUMMARY

According to an embodiment of this disclosure, A cell culture system is provided that includes a cell culture vessel enclosing an interior configured to culture cells in liquid cell culture media, and a media conditioning system. The media conditioning system includes a media conditioning vessel for conditioning liquid media, and an oxygenation column. The oxygenation column includes an enclosure housing an interior space, an oxygen-depleted media inlet fluidly connected to the interior space, an excess gas vent fluidly connected to the interior space, and a lower opening fluidly connected to the interior space. The lower opening is disposed at a lower end of the oxygenation column and in a fluid path between the interior space and the media conditioning vessel. The oxygenation column is designed to mix the oxygen-depleted media with a gas comprising oxygen in a counterflow manner, and thus achieve better dissolved oxygen saturation in the media.

### BRIEF DESCRIPTION OF THE DRAWINGS

FIG. 1 shows a bioreactor system with a media conditioning vessel known in the art.

FIG. 2 shows a schematic of a cell culture system with an oxygenation column, according to one or more embodiments of this disclosure.

FIG. 3 shows an enlarged schematic view of an oxygenation column, according to one or more embodiments of this disclosure.

FIG. 4 shows a close up schematic view a top section of the oxygenation column, according to embodiments.

FIG. 5A shows a media inlet tube from the top plate of the oxygenation column, according to one or more embodiments of this disclosure.

FIG. 5B shows an alternative view of the media inlet tube of FIG. 5A, according to embodiments.

FIG. 6A shows an example of a plurality of media inlet tubes as media fills the top plate, according to embodiments.

FIG. 6B shows an example of a plurality of media inlet tubes as media fills the top plate, according to embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

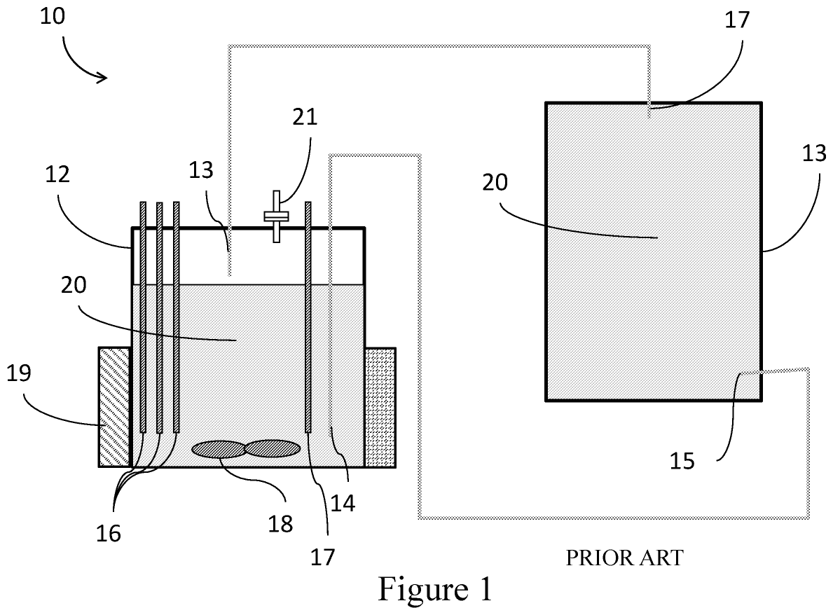

FIG. 1 shows a cell culture system 10 with a conventional media conditioning vessel 12 attached to a bioreactor vessel 13. Media 20 flows out of the media conditioning vessel 12 via the outlet 14 to an inlet 15 of the bioreactor vessel 13. The media 20 within the bioreactor vessel 13 is used to feed cells (not shown) within the bioreactor vessel 13, and may be perfused therethrough to an outlet 17. The media 20 can then, for example, be recirculated to the media conditioning vessel 12 via inlet 11. The media conditioning vessel 12 may be a simple hollow container or vessel (e.g., a glass or plastic beaker or bottle). A complicated system of probes 16 are used to control the composition of the media 20, and one or more stirrers 18 are used to mix the media 20. A temperature control jacket 19 (e.g., water jacket) is shown around the media conditioning vessel 12, which can be used to control temperature of the media 20. The probes 16 can include a combination of sensors and feeds for various components (e.g., oxygen) and may enter the media conditioning vessel 12 through a cap on the vessel body. During operation of the cell culture system 10, oxygen dissolved in the media 20 is consumed by cells located in fixed bed when media 20 is perfused through the bioreactor vessel 13. Media depleted of oxygen is returned to media conditioning vessel 12 where it is reoxygenated and excess of CO₂ is stripped from the media by gas transfer process on the surface of the gas bubbles introduced through the gas sparge 17. Excess gas leaves the media conditioning vessel through a sterile vent filter 21. In high intensity bioprocesses, the oxygen consumption rate in the fixed bed reactor 13 exceeds the oxygenation rate in media conditioning vessel 12. As a result, cell enter an oxygen starvation state, thus decreasing bioprocess efficiency and productivity. To overcome this limitation and to intensify the system performance, embodiments of this disclosure provide an oxygenation system to significantly improve the media oxygenation rate.

Embodiments of this disclosure are directed to media conditioning vessels and systems, and cell culture systems incorporated media conditioning systems. In particular, embodiments of this disclosure provide systems and methods for improving a gas-liquid mass transfer in bioreactors. According to embodiments, an oxygenation column is provided within which cell culture media is exposed to the surface of a packed bed material. The cell culture media enters through a top of the oxygenation column and gas enters the oxygenation column via an inlet in the bottom portion of the column. The gas and liquid are flowed past each other within the oxygenation column. The oxygenation column can include a packed bed of material to increase the surface area of liquid-gas interface and thus enhance gas-liquid mass transfer to re-oxygenate the oxygen-depleted media and to strip excess levels of CO₂ from the media.

Traditionally, one of the difficult parameters to scale in bioprocess is media oxygenation and CO₂ stripping. These parameters depend on multiple primary bioreactor parameters such as mixing, gas flow rates, sparging efficiency, bubble size, bubble residence time. According to embodiments disclosed herein, the oxygenation module enables enhanced gas exchange and operates independently of above listed parameters due to its ability to provide constant increased surface area of liquid-gas interface. The oxygenation column allows an end user to significantly intensify bioprocess in the area of oxygen consumption by cultured cells. This enables higher productivity in comparison to standard bioreactors that rely on gas sparging only.

In some embodiments, it is contemplated that the bioreactor is a fixed bed or packed bed bioreactor with a high density scaffold for cell growth. The system also allows for temperature conditioning of the media and for control of other aspects, such as pH.

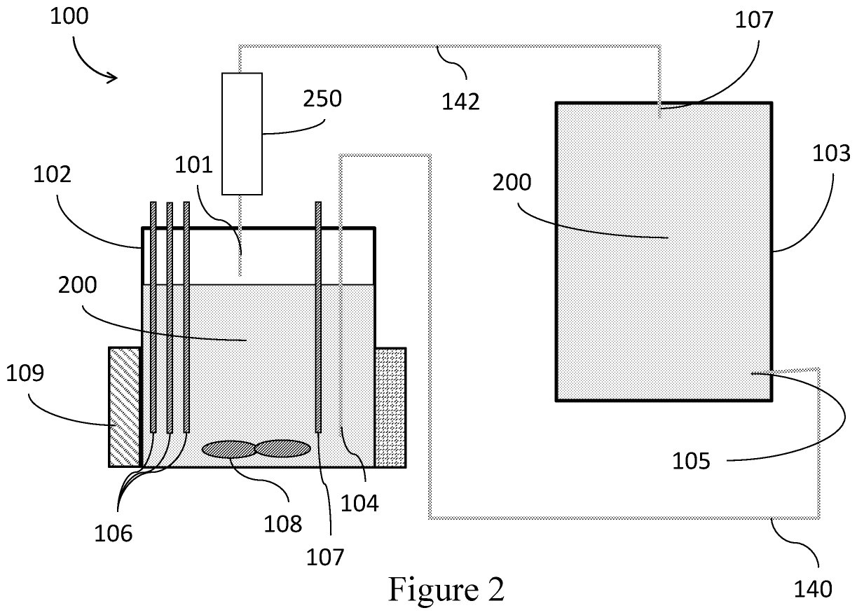

FIG. 2 shows a schematic of a cell culture system 100, according to some embodiments, that includes both a bioreactor vessel 103 for growing cells and/or viral vectors, and a media conditioning system 102 to condition media 200 that is supplied to the bioreactor vessel 103. As shown in FIG. 2, the system 100 can be configured in a recirculation loop, where media 200 flows out of the media conditioning system 102 via on outlet 104 to the bioreactor vessel 103 via tubing 140 or other fluid connector. Within the bioreactor vessel 103, the media supplies necessary nutrients to the cells and maintains a healthy cell environment. The perfused media 200 is then returned to the media conditioning vessel 102 via a return tubing 142 or other connector. The returned media passes through an oxygenation column 250 prior to or while reentering the media conditioning vessel. Embodiments of this disclosure include the entire cell culture system 100, as well as the individual media conditioning system 102 and/or the oxygenation column 250.

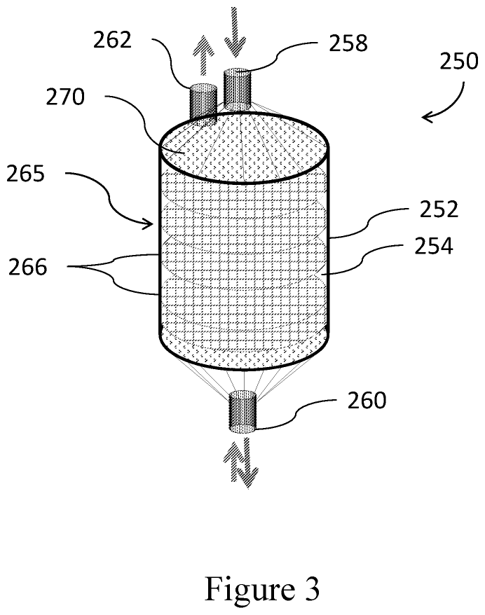

FIG. 3 shows an enlarged schematic view of the oxygenation column 250, according to some embodiments. The oxygenation column 250 includes an enclosure 252 that encloses an interior space 254. Oxygen-depleted media 256 enters the oxygenation column 250 via a media inlet 258, passes through the interior space 254, and exits the oxygenation column 250 via the lower opening 260. Excess gas (e.g., air or oxygen) escaping from the media conditioning vessel 102 enter the oxygenation column 250 via the lower opening 260, passes up through the oxygenation column 250, and exits through the gas vent 262. Thus, a counterflow condition exists within the interior space 254 in which returning media flows down through the oxygenation column 250 while gas flows upward through the oxygenation column 250. The operational principle of the oxygenation column 250 is to increase the surface area of the liquid-gas interface to increase a gas transfer rate from the gas to the liquid (i.e., media) according to equation (1):

$$\frac{d {C}_{L}}{dt} = {k}_{L} a ( {C}_{L}^{*} - {C}_{L} ) \qquad ( 1 )$$

where C_{L} is the oxygen concentration in the media, C_{L}* is the saturation concentration of oxygen in media, t is time, k_{L} is defined as the mass transfer coefficient, and a is the surface area of the media-gas interface.

In aspects of embodiments, the interior space 254 can include a material to increase the surface area of the media-gas interface for improved gas transfer into the media. For example, the oxygenation column can be packed with a highly porous material 265. It is contemplated that this highly porous material can take a variety of forms, thus embodiments are not limited to a particular porous material. The porous material can be a polymer, metal, ceramic, glass, or other suitable material compatible with bioprocessing applications. In some preferred embodiments, the porous material 265 includes polyethylene terephthalate (PET). The porous material can be in the form of PET sheets that are stacked (see FIG. 3) or wound into a roll within the oxygenation column 250, according to examples of embodiments. The PET sheets can be formed of a mesh material having an ordered array of fibers defining and a plurality of openings therebetween in an ordered array. In embodiments, the mesh is a woven mesh of one or more PET fibers, or is 3D printed, or is a stamped or molded mesh material.

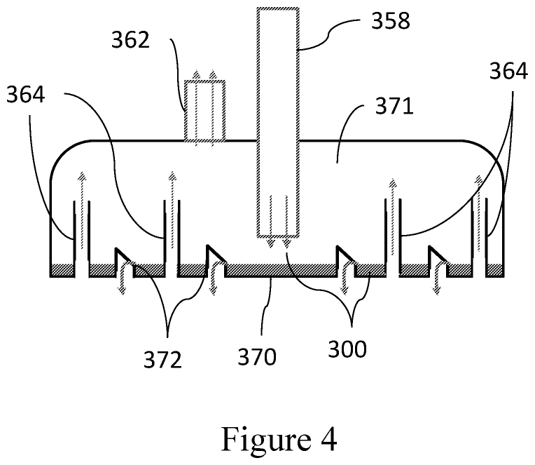

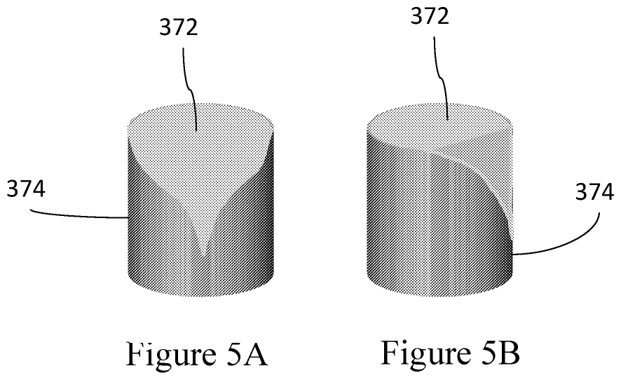

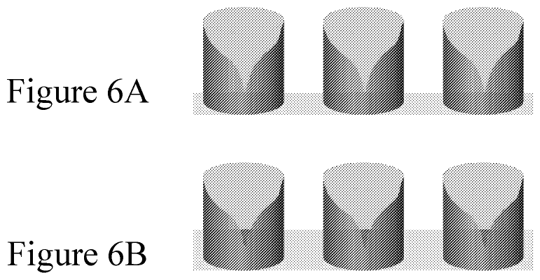

The oxygenation column can include a top plate 270, as shown in FIG. 3. FIG. 4 shows an enlarged view of an example of a top plate 370, according to some embodiments. As shown in FIG. 4, the media inlet 358 feeds oxygen-depleted media into the oxygenation column and to the top plate 370, while gas vent 362 vents excess gas out of the top of the oxygenation column. One or more gas outlets 364 inside the packed oxygenation column allow the excess gas from within the oxygenation column to rise to a head space 371 above the top plate 370. The media 300 entering via the media inlet 358 falls to the top plate 370 where it pools before rising to a level sufficient to enter one or more media inlet openings 372. After rising to the level of the opening in the media inlet openings 372, the media 300 flows through the top plate 370 via the media inlet openings 372 and into the interior of the oxygenation column that contains a packed bed of porous material. Multiple media inlet openings 372 and gas outlets 364 can be provided to improve uniformity of media entering into and gas exiting the packed bed zone. The media inlet openings 372 are configured to equalize media flow across the top plate 370 and equally distribute media flow among all media inlet openings 372 in the top plate 370. For example, the media inlet opening 372 can include a media inlet tube 374, as shown in FIGS. 5A and 5B, with a slanted cut-out flow opening in the wall of the media inlet tube 374. The flow opening becomes wider toward the top of the media inlet tube 374. As a result of this, the flow through each media inlet tube 374 is proportional to the liquid level in top plate, as shown in FIGS. 6A and 6B. Horizontal placement of top flow distribution plate will provide a condition for self-regulated uniform flow distribution through the flow inlet tubes 374.

According to embodiments, the oxygenation column 250, 350 can be an add-on component that can be attached after-market to media conditioning vessels. In other embodiments, the oxygenation column can be integrated with or pre-installed on the media conditioning vessel.

Optionally, the media conditioning vessel 102 is temperature controlled. That is, the temperature of the space within the vessel 102 can be heated or cooled control the temperature of gas and/or media that passes into or through the gas media conditioning vessel 102 or the oxygenation column 250. In some embodiments, the temperature control is used to heat or cool the gas, which then heats or cools the media within the media conditioning vessel 102 or the oxygenation column. Optionally, this temperature control can be achieved by an integrated temperature control device 109, such as, for example, a heater or cooler. Alternatively, the gas can be temperature controlled prior to entering the enclosure.

The bioreactor system 100 can also include one or more sensors 106 for detecting qualities of the gas and/or media. The sensors 106 can be provided in the media conditioning vessel 102, as shown in FIG. 2. However, sensors can also be supplied in line with the media, either up or down stream of the media inlet 101 and media outlet 104, or can be supplied in the bioreactor vessel itself, or can be supplied in the oxygenation column 250, 350. Based on feedback from the sensors, the components of the gas via inlet and/or the temperature control device can be controlled to achieve the desired composition and temperature of the cell culture media for optimal performance of the culture. For example, gas composition can be adjusted to achieve a desired O₂ and/or CO₂ level; and pH can be adjusted.

The enclosure 109 for the media conditioning vessel 102 can be a thermal enclosure. As used herein, “thermal enclosure” means that the enclosure is thermally insulated so a temperature in the interior of the thermal enclosure can be more easily controlled. As an alternative or in addition to a thermal enclosure, the enclosure 102 can include a heat source for controlling a temperature within the enclosure, or a temperature of a gas within the enclosure, as discussed above. The heat source may be an integral structure and function of the enclosure 109, or it could be a separate component that the enclosure 109 is configured to accept, as needed. According to some embodiments, the heat source provides heat to the gas that is sufficient to control a temperature of the media in the media exchange system within a desired range for cell culture.

In some embodiments, the media conditioning system further includes one or more sensors for sensing a property of the gas within the enclosure, or of the media within the oxygenation column, or of the media prior to entering, after exiting, or while within the bioreactor. The one or more sensors can measure temperature, pH, oxygen (O₂), CO₂, or any of a number of variables that are relevant to the cell culture operation being performed.

There are many advantages to the media conditioning systems and vessels disclosed herein. For example, the design allows the media conditioning vessel to be a single-use or disposable vessel. The reusable or single use components of the system can reduce cost of operation, while achieving gas transport to the media using the oxygenation column. The gas can also be temperature controlled in the system, which thus effectively controls the temperature of the media. In addition, due to the possible embodiments, the system can handle large amounts of media, and do so efficient. Further, the simplified design can avoid the use of adhesives or high-particulate materials, thus avoiding potential complications or undesired components in the bioprocessing industry.

It is contemplated that the cell culture system may be used with a bioreactor having a packed-bed of fixed-bed cell culture substrate. In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Media flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to some embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

The cell culture substrate can be a woven mesh layer made of a first plurality of fibers running in a first direction and a second plurality of fibers running in a second direction. The woven fibers of the substrate form a plurality of openings, which can be defined by one or more widths or diameters. The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Without wishing to be bound by theory, it is believed that the three-dimensional structure of the substrate is advantageous as it provides a large surface area for culturing adherent cells, and the structural rigidity of the mesh can provide a consistent and predictable cell culture matrix structure that enables uniform fluid flow.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix is comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

As described herein, the same material used for the cell culture substrate can also be used in the packed bed zone of the oxygenation column 250, 350.

The system 100 of FIG. 2 includes a bioreactor 103 housing the cell culture matrix of one or more embodiments disclosed herein. The bioreactor 103 can be fluidly connected to a media conditioning vessel 102, as described above, and the system is capable of supplying a cell culture media within the conditioning vessel 102 to the bioreactor 103. The media conditioning vessel 102 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 102 can also contain a pump or an impeller for media mixing. All media parameters measured by sensors listed above can be controlled by a media conditioning control unit in communication with the media conditioning vessel 102, and capable of measuring and/or adjusting the conditions of the cell culture media to the desired levels. As shown in FIG. 2, the media conditioning vessel 102 is provided as a vessel that is separate from the bioreactor vessel 103. This can have advantages in terms of being able to condition the media separate from where the cells are cultured, and then supplying the conditioned media to the cell culture space. However, in some embodiments, media conditioning can be performed within the bioreactor vessel 103.

The media from the media conditioning vessel 102 is delivered to the bioreactor 103 via an connector or tubing 140, which may also include an injection port for cell inoculum to seed and begin culturing of cells. The bioreactor vessel 103 may also include on or more outlets to another connector or tubing 142 through which the cell culture media exits the vessel 103. To analyze the contents of the outflow from the bioreactor 103, one or more sensors may be provided in the line. In some embodiments, the system 100 includes a flow control unit for controlling the flow into and/or out of the bioreactor 103 and/or media conditioning system 100. For example, the flow control unit may receive a signal from the one or more sensors (e.g., an 02 sensor) and, based on the signal, adjust the flow into the bioreactor 103 by sending a signal to a pump (e.g., peristaltic pump) upstream of the inlet to the bioreactor 103. Thus, based on one or a combination of factors measured by the sensors, the pump can control the flow into the bioreactor 103 to obtain the desired cell culturing conditions.

The media perfusion rate is controlled by the signal processing unit that collects and compares sensors signals from media conditioning system 100 and sensors located, for example, within or at the outlet of the bioreactor 103. Because of the pack flow nature of media perfusion through the packed bed bioreactor, nutrients, pH and oxygen gradients are developed along the packed bed. The perfusion flow rate of the bioreactor can be automatically controlled by the flow control unit operably connected to the peristaltic pump.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a cell culture system comprising a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and a media conditioning system comprising: a media conditioning vessel configured for conditioning liquid media; and an oxygenation column, the oxygenation column comprising an enclosure housing an interior space, an oxygen-depleted media inlet fluidly connected to the interior space, an excess gas vent fluidly connected to the interior space, and a lower opening fluidly connected to the interior space, wherein the lower opening is disposed at a lower end of the oxygenation column and in a fluid path between the interior space and the media conditioning vessel, and wherein the oxygenation column is configured to mix the oxygen-depleted media with a gas comprising oxygen in a counterflow manner.

Aspect 2 pertains to the cell culture system of Aspect 1, wherein the oxygen-depleted media inlet is disposed at an upper end of the oxygenation column and in a fluid path to receive media from the interior of the bioreactor vessel.

Aspect 3 pertains to the cell culture system of Aspect 1 or 2, wherein the excess gas vent is disposed at the upper end of the oxygenation module.

Aspect 4 pertains to the cell culture system of Aspects 1-3, wherein the lower opening is configured to supply oxygen-rich gas to the interior space and to supply media from the oxygenation column to the media conditioning vessel.

Aspect 5 pertains to the cell culture system of Aspects 1-4, wherein the oxygenation column comprises a porous material in the interior space.

Aspect 6 pertains to the cell culture system of Aspect 5, wherein the porous material is configured to increase an area of gas-media interface in the oxygenation column.

Aspect 7 pertains to the cell culture system of Aspect 5 or 6, wherein the porous material is a packed bed of the porous material.

Aspect 8 pertains to the cell culture system of any of Aspects 5-7, wherein the porous material is the same material used as a cell culture substrate disposed in the interior of the bioreactor vessel.

Aspect 9 pertains to the cell culture system of any of Aspect 1-8, wherein the porous material comprises an ordered physical structure comprising an array of pores.

Aspect 10 pertains to the cell culture system of Aspect 9, wherein the porous material comprises a woven mesh material.

Aspect 11 pertains to the cell culture system of Aspect 9 or 10, wherein the porous material comprises a plurality of sheets of the porous material in a stacked arrangement within the interior space.

Aspect 12 pertains to the cell culture system of any of Aspects 1-11, wherein the cell culture system is arranged in a perfusion loop.

Aspect 13 pertains to the cell culture system of any of Aspects 1-12, wherein the oxygenation module is configured to passively regulate flow of oxygen-depleted media through the interior space based on a rate at which oxygen-depleted media fills a top plate of the oxygenation column.

Aspect 14 pertains to the cell culture system of Aspect 13, wherein the top plate comprises one or more openings, the one or more opening being configured to allow a higher rate of flow of the oxygen-depleted media through the one or more openings as a height of the oxygen-depleted media on the top plate increase.

Aspect 15 pertains to the cell culture system of Aspect 13 or 14, wherein the one or more openings comprising a media inlet tube rising into the top plate into a head space above the top plate.

Aspect 16 pertains to the cell culture system of Aspect 15, wherein the media inlet tube comprises a media inlet opening configured to allow the flow of oxygen-depleted media from the head space above the top plate to below the top plate.

Aspect 17 pertains to the cell culture system of Aspect 16, wherein the media inlet opening comprises a variable width.

Aspect 18 pertains to the cell culture system of Aspect 16 or 17, wherein a width of the media inlet opening is narrower at a bottom of the media inlet tube than at a top of the media inlet tube.

Aspect 19 pertains to the cell culture system of any of Aspects 16-18, wherein the width of the media inlet opening increases along a height of the media inlet tube from the bottom of the media inlet tube.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A cell culture system comprising:
a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and
a media conditioning system comprising:
a media conditioning vessel configured for conditioning liquid media; and
an oxygenation column, the oxygenation column comprising an enclosure housing an interior space, an oxygen-depleted media inlet fluidly connected to the interior space, an excess gas vent fluidly connected to the interior space, and a lower opening fluidly connected to the interior space,
wherein the lower opening is disposed at a lower end of the oxygenation column and in a fluid path between the interior space and the media conditioning vessel,
wherein the oxygen-depleted media inlet is disposed at an upper end of the oxygenation column and in a fluid path to receive media from the interior of the bioreactor vessel,
wherein the excess gas vent is disposed at the upper end of the oxygenation module, and
wherein the oxygenation column is configured to mix the oxygen-depleted media with a gas comprising oxygen in a counterflow manner.

2. (canceled)

3. (canceled)

4. The cell culture system of claim 1, wherein the lower opening is configured to supply oxygen-rich gas to the interior space and to supply media from the oxygenation column to the media conditioning vessel.

5. The cell culture system of claim 1, wherein the oxygenation column comprises a porous material in the interior space, the porous material being configured to increase an area of gas-media interface in the oxygenation column.

6. (canceled)

7. (canceled)

8. The cell culture system of claim 5, wherein the porous material is the same material used as a cell culture substrate disposed in the interior of the bioreactor vessel.

9. The cell culture system of claim 1, wherein the porous material comprises an ordered physical structure comprising an array of pores.

10. The cell culture system of claim 9, wherein the porous material comprises a woven mesh material.

11. The cell culture system of claim 9, wherein the porous material comprises a plurality of sheets of the porous material in a stacked arrangement within the interior space.

12. The cell culture system of claim 1, wherein the cell culture system is arranged in a perfusion loop.

13. The cell culture system of claim 1, wherein the oxygenation module is configured to passively regulate flow of oxygen-depleted media through the interior space based on a rate at which oxygen-depleted media fills a top plate of the oxygenation column.

14. The cell culture system of claim 13, wherein the top plate comprises one or more openings, the one or more opening being configured to allow a higher rate of flow of the oxygen-depleted media through the one or more openings as a height of the oxygen-depleted media on the top plate increase.

15. The cell culture system of claim 13, wherein the one or more openings comprising a media inlet tube rising into the top plate into a head space above the top plate.

16. The cell culture system of claim 15, wherein the media inlet tube comprises a media inlet opening configured to allow the flow of oxygen-depleted media from the head space above the top plate to below the top plate.

17. The cell culture system of claim 16, wherein the media inlet opening comprises a variable width.

18. The cell culture system of claim 16, wherein a width of the media inlet opening is narrower at a bottom of the media inlet tube than at a top of the media inlet tube.

19. The cell culture system of claim 16, wherein the width of the media inlet opening increases along a height of the media inlet tube from the bottom of the media inlet tube.

20. An oxygenation column for oxygenating cell culture media, the oxygenation column comprises:
an enclosure housing an interior space;
an oxygen-depleted media inlet fluidly connected to the interior space;
an excess gas vent fluidly connected to the interior space; and
a lower opening fluidly connected to the interior space,
wherein the lower opening is disposed at a lower end of the oxygenation column and in a fluid path between the interior space and an exterior of the oxygenation column, and
wherein the oxygenation column is configured to mix oxygen-depleted media with a gas comprising oxygen in a counterflow manner.

21. The oxygenation column of claim 20, wherein the oxygen-depleted media inlet is disposed at an upper end of the oxygenation column and in a fluid path to receive media from the interior of the bioreactor vessel.

22. The oxygenation column of claim 20, wherein the excess gas vent is disposed at the upper end of the oxygenation module.

23-26. (canceled)

27. The oxygenation column of claim 20, wherein the porous material comprises a woven mesh material an ordered physical structure comprising an array of pores, and
wherein the porous material comprises a plurality of sheets of the porous material in a stacked arrangement within the interior space.

28. (canceled)

29. (canceled)

30. The oxygenation column of claim 20, wherein the oxygenation module is configured to passively regulate flow of oxygen-depleted media through the interior space based on a rate at which oxygen-depleted media fills a top plate of the oxygenation column,
wherein the top plate comprises one or more openings, the one or more opening being configured to allow a higher rate of flow of the oxygen-depleted media through the one or more openings as a height of the oxygen-depleted media on the top plate increase,
wherein the one or more openings comprising a media inlet tube rising into the top plate into a head space above the top plate
wherein the media inlet tube comprises a media inlet opening configured to allow the flow of oxygen-depleted media from the head space above the top plate to below the top plate, and
wherein the media inlet opening comprises a variable width.

31-36. (canceled)
